(19) United States
(12) Patent Application Publication
(10) Pub. No.: US 2020/0297997 A1
(43) Pub. Date: Sep. 24, 2020

(54) Mechanical Coupling Of An Analyte-Selective Sensor And An Infusion System And Information Conveyance Between The Same

(71) Applicant: Biolinq, Inc., San Diego, CA (US)

(72) Inventors: Joshua Windmiller, Del Mar, CA (US); Jared Rylan Tangney, Encinitas, CA (US); Thomas Arnold Peyser, Menlo Park, CA (US)

(73) Assignee: Biolinq, Inc., San Diego, CA (US)

(21) Appl. No.: 16/899,541

(22) Filed: Jun. 11, 2020

Related U.S. Application Data
(63) Continuation-in-part of application No. 16/824,700, filed on Mar. 20, 2020.
Application No. 16/824,700 is a continuation-in-part of application No. 16/666,259, filed on Oct. 28, 2019.
Application No. 16/666,259 is a continuation of application No. 16/152,372, filed on Oct. 4, 2018, now Pat. No. 10,492,708.
Application No. 16/152,372 is a continuation of application No. 15/590,105, filed on May 9, 2017, now Pat. No. 10,092,207.
(60) Provisional application No. 62/823,628, filed on Mar. 25, 2019.
(60) Provisional application No. 62/336,724, filed on May 15, 2016.
(60) Provisional application No. 62/862,658, filed on Jun. 17, 2019.

(51) Int. Cl. A61N 1/30; A61N 1/05; A61B 5/1468; A61B 5/05; A61B 5/145; A61M 5/172

(52) U.S. Cl. CPC A61N 1/30; A61N 1/05; A61B 5/1468; A61B 2562/125; A61B 5/14546; A61M 5/1723; Y02E 60/527; A61B 5/05

(57) ABSTRACT

A device and method for the coupling of an analyte-selective sensor and an infusion system into a singular body-worn device is disclosed herein. Following the coupling, information and/or power is/are exchanged between the two modalities (analyte-selective sensor and an infusion system) by means of a wireless electromagnetic transmission or an electrical connector. The analyte-selective sensor is configured to penetrate the stratum corneum to access the viable epidermis or dermis and measure the presence of an analyte. The infusion system is configured to penetrate the stratum corneum and deliver a solution-phase therapeutic agent.

### BACKGROUND OF THE INVENTION

### Field of the Invention

The present invention generally relates to therapeutic delivery mechanisms, analyte-selective sensors and methods for configuration of the same.

### Description of the Related Art

The continuous delivery of therapeutic agents remains an important technology in modern medical devices. The most important example of such medical devices are insulin pumps, also known as continuous subcutaneous insulin infusion (CSII) systems, which are widely used by individuals with insulin-dependent diabetes mellitus. Insulin pumps were developed in the 1980's and commercialized in the 1990's to provide a more physiological method of insulin delivery than the infrequent subcutaneous injection of insulin by syringe and hypodermic needle. The importance of improved methods of insulin delivery was further recognized in the aftermath of the publication of the Diabetes Control and Complication Trial (DCTT) in 1992 which showed that intensive insulin therapy dramatically reduced the incidence and severity of long-term complications of diabetes. More recently, insulin pumps have been configured to automatically suspend insulin infusion in the event of actual or impending hypoglycemia as determined by a continuous glucose monitoring system. Insulin pumps have also been configured to modulate insulin delivery continuously in response to glucose levels measured by continuous glucose monitoring systems. In these scenarios, both the analyte sensing and therapeutic delivery modalities comprise two distinct and extricable devices, both of which are worn on the body. However, in line with aims towards integrated sense-treat systems and the desire for miniaturized body-worn devices, the integration of both the analyte sensing and therapeutic delivery constituents into a singular wearable device is an active area of development. A major obstacle for many patients in using these technologies, however, is the use of two separate on-body devices, as shown in FIG. 2 with two devices 205 and 210 positioned on a user 215. In line with aims towards integrated sense-treat systems and the desire for miniaturized body-worn devices, the co-location of both the analyte sensing and therapeutic delivery constituents into a singular wearable device is an active area of development. With the above being said, the integration of sensing and therapy contingents presents its own set of unique challenges, namely, developing robust methods for mechanical coupling and electrical interface between these two extricable components that are configured to operate as a singular body-worn device. In light of the challenges associated with the integration of both elements, which might be coupled using different methods into a single body-worn device, the sensing and delivery components have often been embodied by distinct and non-couplable components. In such embodiments, the user is relegated to applying and wearing said distinct components in spatially distinct locations on the body.

Prior art solutions have largely been concerned with operating both sensing and delivery systems as distinct body-worn entities that are spatially separated by a sufficient extent so as to avoid the challenges associated with the insertion of two cannulae physically attached to a single integrated device. Additionally, implementation of the analyte sensing and therapeutic delivery modalities as physically distinct devices so that these contingents can be worn in different regions resolves the challenge of undesired interactions between the two systems. Such interactions can take multiple forms—cross-talk, interference, contamination, and dilution—which impact the performance, accuracy, and reliability of the sensing routine. Prior art embodiments of the analyte sensing modality include cannula-assisted, subcutaneously-implanted wire-based sensors configured to quantify an analyte using electrochemical transduction techniques; continuous glucose monitoring systems are one example. Prior art embodiments of the therapeutic delivery modality include cannula-based patch pumps and infusion sets configured to deliver a therapeutic agent to the subcutaneous adipose tissue (subcutis) by means of application of a pressure on a reservoir containing the therapy in the solution phase; insulin infusion systems are one example. FIG. 1A is a prior art needle-/cannula-based analyte-selective sensor 110 with a user interface device 115 and mobile phone 105 configured for the quantification of glucose in the subcutaneous adipose tissue. FIG. 1B is a prior art needle-/cannula-based analyte-selective sensor 130 with a user interface device 125 configured for the quantification of glucose in the subcutaneous adipose tissue. FIG. 1C is a prior art needle-/cannula-based analyte-selective sensor 150 with a user interface device 145 configured for the quantification of glucose in the subcutaneous adipose tissue. More recent prior art has instructed of the co-location of both sensing and delivery modalities within a single body-worn device, albeit both modalities in such prior art are inextricable. In this manner, should the supply of therapeutic agent within the therapeutic delivery contingent become depleted, the entire system, including the analyte-sensing modality, must be removed and replaced despite the fact that the analyte-sensing modality might still engender many days of remaining useful lifetime. In another embodiment, the analyte-selective sensor can be replaced with the same frequency as the infusion system by reducing the sensor to a minimal number of components while taking advantage of the circuitry and power source residing in the said infusion system.

Van Antwerp et al., U.S. Pat. No. 9,968,742, discloses a dual insertion set for supplying a fluid to the body of a patient and for monitoring a body characteristic of the patient.

Curtis, U.S. Pat. No. 9,199,582 discloses a method and system for providing an integrated analyte monitoring system and on-body patch pump with multiple cannulas and a sensor combination.

Gym, U.S. Patent Publication Number 20120184909 discloses a base part for a medication delivery device.

Regittnig, U.S. Patent Publication Number 20140288399 discloses a medical apparatus for supplying a medication fluid into an organism and for detecting a substance of the organism.

Geismar et al., U.S. Patent Publication Number 2006017761 discloses a dual insertion set that includes a base, an infusion portion, a sensor portion, and at least one piercing member.

Yodat et al, U.S. Pat. No. 9,056,161 discloses a system and a method for delivering fluid to and sensing analyte levels in the body of the patient.

Ward et al., U.S. Patent Publication Number 20160354542 discloses the concept, and method of creating, a dual use device intended for persons who take insulin.

O'Connor et al., U.S. Patent Publication Number 20170173261 discloses systems and methods for automatically delivering medication to a user.

Current needle- and cannula-based infusion systems, configured for the delivery of a solution-phase therapeutic agent (i.e. insulin) are often paired with needle- and cannula-based sensor systems configured for continuous quantification of an analyte (i.e. glucose). Although such systems operate in unison and are configured to operate as distinct components (sensing and delivery), both systems have yet to be implemented as a single body-worn device. Although this is partly due to challenges associated with the insertion of two cannulae physically attached to a single integrated device, the primary challenge arises due to the mechanical coupling of both systems while simultaneously supporting a method for information conveyance between the two systems. Namely, the coupling of the analyte sensing and therapeutic delivery contingents requires a method for the retention of said components into a singular body-worn device while facilitating the conveyance of information and/or power either unidirectionally or bidirectionally between said components (or routed through an intermediary information processing device). In all embodiments, information and power are considered to constitute electromagnetic quantities. In addition, current advances in analyte-selective sensors, such as continuous glucose monitors, have enabled the commercialization of devices featuring wear lifetimes between seven and fourteen days. However, current therapeutic delivery mechanisms, such as insulin infusion systems, are only capable of accommodating a three day supply of therapeutic agent onboard, thereby implying that the therapeutic delivery contingent will require replacement well in advance of the conclusion of the functional lifetime of the analyte-selective sensor. Should both systems be integrated into a singular body-worn contingent, as proposed in the prior art, this would mandate that both systems be removed concurrently from the skin and replaced to replenish the internal supply of therapeutic agent within the therapeutic delivery contingent, thereby resulting in a concession in useful lifetime engendered by the analyte-selective sensor. There is a need for a solution to the problem of the incommensurate durations of use of current glucose sensors and insulin infusion systems.

### BRIEF SUMMARY OF THE INVENTION

The current invention teaches of methods for the coupling of extricable analyte sensing and therapeutic delivery modalities that is assembled by a user to create a singular body-worn device configured in open-loop or closed-loop embodiments. The coupling operation is executed, in a straightforward manner, by a user either prior to application to the skin of the user or in sequence following application of one of the modalities (sensing or delivery). Following coupling, information and/or power is/are exchanged between the two modalities by means of a wireless electromagnetic transmission or an electrical connector. Alternatively, information may be transferred between the two modalities by means of an electromagnetic interaction with an intermediary electronic device. The intermediary electronic device, in one embodiment, retains both the analyte sensing and therapeutic delivery modalities and, in another embodiment, constitutes a separate and physically distinct information processing device such as a smartphone, smartwatch, tablet, computer, or other body-worn device. Embodiments of the entire system either include an open-loop system, whereby the wearer adjusts dosing of the therapeutic intervention based on levels of the analyte, or plurality of analytes, and a close-loop system, whereby a control algorithm autonomously adjusts dosing of the therapeutic intervention or plurality of therapeutic interventions. The invention disclosed herein permits the replacement of a therapeutic delivery contingent, such as an insulin infusion system, while the analyte-sensing contingent remains unperturbed and continues with the sensing operation. In this manner, the user can realize the full functional lifetime of the analyte sensor, not to mention the therapeutic delivery contingent. The current invention instructs of a facile method for mechanical coupling and de-coupling of the therapeutic delivery contingent from the analyte-selective sensor while simultaneously facilitating electromagnetic interface between the same for the purpose of information or power transfer.

One aspect of the present invention is a method for the coupling of an analyte-selective sensor and an infusion system into a singular body-worn device. The method includes firstly, positioning an analyte-selective sensor on the skin of a wearer. The analyte-selective sensor is configured to penetrate the stratum corneum to access the viable epidermis or dermis and measure the presence of an analyte or plurality of analytes in a selective fashion. It is generally understood that selective fashion means the ability of an analyte-selective sensor to measure at least one analyte of interest while mitigating the deleterious signal contributions imparted by co-circulating endogenous (i.e. metabolites, ions, proteins) and exogenous (i.e. pharmacologic agents, therapeutic agents) electroactive compounds that occupy the biological milieu. The method also includes secondly, positioning an infusion system on the skin of a wearer. The infusion system is configured to penetrate the stratum corneum and deliver, in a controlled fashion, a solution-phase therapeutic agent or collection of therapeutic agents to a physiological compartment beneath the dermis. It is generally understood that controlled fashion means the ability of an infusion system to deliver a specified dosage, concentration, or quantity of therapeutic agent; this can either comprise bolus delivery, wherein said therapeutic agent is given in a brief time duration, or basal delivery, wherein said therapeutic agent is given over an extended duration of time. The positioning requires a mechanical retention between the infusion system and the analyte-selective sensor to form a singular body-worn device. The method also includes lastly, conveyance of electromagnetic energy between the analyte-selective sensor and the infusion system to effectuate a transaction between the analyte-selective sensor and the infusion system.

Another aspect of the present invention is a method for the coupling of an analyte-selective sensor and an infusion system into a singular body-worn device. The method includes firstly, positioning the infusion system on the skin of a wearer. The infusion system is configured to penetrate the stratum corneum and deliver, in a controlled fashion, a solution-phase therapeutic agent or collection of therapeutic agents to a physiological compartment beneath the dermis. The method also includes secondly, positioning the analyte-selective sensor on the skin of a wearer. The analyte-selective sensor is configured to penetrate the stratum corneum to access the viable epidermis or dermis and measure the presence of an analyte or plurality of analytes in a selective fashion. The positioning requires a mechanical retention between the analyte-selective sensor and the infusion system to form a singular body-worn device. The method also includes lastly, conveyance of electromagnetic energy between the analyte-selective sensor and the infusion system to effectuate a transaction between the analyte-selective sensor and infusion system.

Yet another aspect of the present invention is a method for the coupling of an analyte-selective sensor configured to penetrate the stratum corneum to access the viable epidermis or dermis and measure the presence of an analyte or plurality of analytes in a selective fashion and an infusion system configured to penetrate the stratum corneum and deliver, in a controlled fashion, a solution-phase therapeutic agent or collection of therapeutic agents to a physiological compartment beneath the dermis. The method includes firstly, engaging a mechanical retention mechanism between the analyte-selective sensor and the infusion system to form a singular device. The method also includes secondly, positioning the singular device on the skin of a wearer. The method also includes lastly, conveyance of electromagnetic energy between the analyte-selective sensor and the infusion system to effectuate a transaction between the analyte-selective sensor and infusion system.

Yet another aspect of the present invention is a method for the coupling of an analyte-selective sensor configured to penetrate the stratum corneum to access the viable epidermis or dermis and measure the presence of an analyte or plurality of analytes in a selective fashion and an infusion system configured to penetrate the stratum corneum and deliver, in a controlled fashion, a solution-phase therapeutic agent or collection of therapeutic agents to a physiological compartment beneath the dermis. The method includes firstly, engaging a mechanical retention mechanism between an intermediary apparatus and both the analyte-selective sensor and the infusion system to form a singular device. The method also includes secondly, positioning the singular device on the skin of a wearer. The method also includes lastly, conveyance of electromagnetic energy between the analyte-selective sensor and the infusion system to effectuate a transaction between the analyte-selective sensor and the infusion system. It is generally understood that said intermediary apparatus or device is configured with two geometric features in which said analyte-selective sensor and infusion system may be retained or otherwise mechanically coupled so as to render both devices in a fixed position with respect to one another. In one embodiment, the said intermediary apparatus is purely a mechanical apparatus to couple said analyte-selective sensor and infusion system into a singular integrated device; no embedded electronic systems are featured in said intermediary apparatus. In another embodiment, the said intermediary apparatus contains an embedded electronic system capable of the conveyance of electromagnetic energy (i.e. power, data) between the analyte-selective sensor and infusion system.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1A is a prior art needle-/cannula-based analyte-selective sensors configured for the quantification of glucose in the subcutaneous adipose tissue.

FIG. 1B is a prior art needle-/cannula-based analyte-selective sensors configured for the quantification of glucose in the subcutaneous adipose tissue.

FIG. 1C is a prior art needle-/cannula-based analyte-selective sensors configured for the quantification of glucose in the subcutaneous adipose tissue.

FIG. 2 is a prior art embodiment of an analyte-selective sensor device (left) and an infusion system (right), both devices featuring extensive spatial separation circumvent undesired interactions.

FIG. 3 is a prior art needle-/cannula-based analyte-selective sensor (left) configured for the quantification of glucose in the subcutaneous adipose tissue and a microneedle array-based analyte-selective sensor (right) configured for the quantification of glucose in the dermis.

FIG. 4 is a pictorial representation (not to scale) of an infusion system configured to operate within the subcutaneous tissue (left) and an analyte-selective sensor configured to operate within the dermis (right), with both located in close spatial proximity.

FIG. 5A is an illustration of an integration of a microneedle array-based analyte-selective sensor into an infusion set.

FIG. 5B is an integration of a microneedle array-based analyte-selective sensor into a patch pump.

FIG. 6A is an integration of a microneedle array-based analyte-selective sensor into a patch pump.

FIG. 6B is an integration of a microneedle array-based analyte-selective sensor into a patch pump.

FIG. 6C is an isolated view of circle 6C of FIG. 6B.

FIG. 7A is a top perspective view of a microneedle array-based analyte-selective sensor featuring six electrical pads to transfer power and/or information between the therapeutic delivery system (not shown) and analyte-selective sensor.

FIG. 7B is a top plan view of a microneedle array-based analyte-selective sensor featuring six electrical pads to transfer power and/or information between the therapeutic delivery system (not shown) and analyte-selective sensor.

FIG. 7C is a side elevation view of a microneedle array-based analyte-selective sensor.

FIG. 7D is a bottom plan view of a microneedle array-based analyte-selective sensor.

FIG. 7E is a bottom perspective view of a microneedle array-based analyte-selective sensor.

FIG. 8A is a top plan view of a therapeutic delivery system featuring a cavity designed to retain and electromagnetically interface with the analyte-selective sensor (not shown) featured in FIGS. 7A-7E.

FIG. 8B is a side elevation view of a therapeutic delivery system.

FIG. 8C is a bottom plan view of a therapeutic delivery system featuring a cavity designed to retain and electromagnetically interface with the analyte-selective sensor (not shown) featured in FIGS. 7A-7E.

FIG. 8D is a bottom plan view of a therapeutic delivery system featuring the analyte-selective sensor featured in FIGS. 7A-7E and a cannula.

FIG. 9A is a side perspective view of a therapeutic delivery system with a needle shown.

FIG. 9B is front elevation view of a therapeutic delivery system with the needle shown.

FIG. 9C is side perspective view of a therapeutic delivery system with a cannula shown.

FIG. 9D is rear elevation view of a therapeutic delivery system with the cannula shown.

FIG. 10 is an illustration of an application of the analyte-selective sensor to the skin of a wearer.

FIG. 10A is an illustration of an application of the therapeutic delivery system on the posterior of the analyte-selective sensor.

FIG. 10B is an illustration of the mechanically- and electromagnetically-coupled analyte-selective sensor and therapeutic delivery system on the skin of a wearer.

FIG. 11 is a side perspective view of the mechanically- and electromagnetically-coupled analyte-selective sensor and therapeutic delivery system with the microneedle array of the said analyte-selective sensor and the cannula/needle of the said therapeutic delivery system detailed.

FIG. 12 is a flow chart of method for coupling of an analyte-selective sensor and an infusion system into a singular body-worn device.

FIG. 13 is a flow chart of method for coupling of an infusion system and an analyte-selective sensor into a singular body-worn device.

FIG. 14 is a flow chart of method for coupling of an analyte-selective sensor and an infusion system into a singular body-worn device prior to skin application.

FIG. 15 is a flow chart for a method for the coupling of an analyte-selective sensor and infusion system, facilitated by an intermediary apparatus, prior to skin application.

FIG. 16 is a flow chart for a method of an OPEN LOOP embodiment.

FIG. 17 is a flow chart for a method of a CLOSED LOOP embodiment.

FIG. 18 is a block/process flow diagram illustrating the inputs, outputs, and major constituents under the OPEN LOOP embodiment.

FIG. 19 is a block/process flow diagram illustrating the inputs, outputs, and major constituents under the CLOSED LOOP embodiment.

FIG. 20 is a cross-sectional view of an intermediary device for coupling of an infusion system and an analyte-selective sensor into a singular body-worn device.

FIG. 21 is a cross-sectional view of an intermediary device integrated with an infusion system and an analyte-selective sensor into a singular body-worn device.

FIG. 22 is a cross-sectional view of an alternative embodiment of an intermediary device for coupling of an infusion system and an analyte-selective sensor into a singular body-worn device.

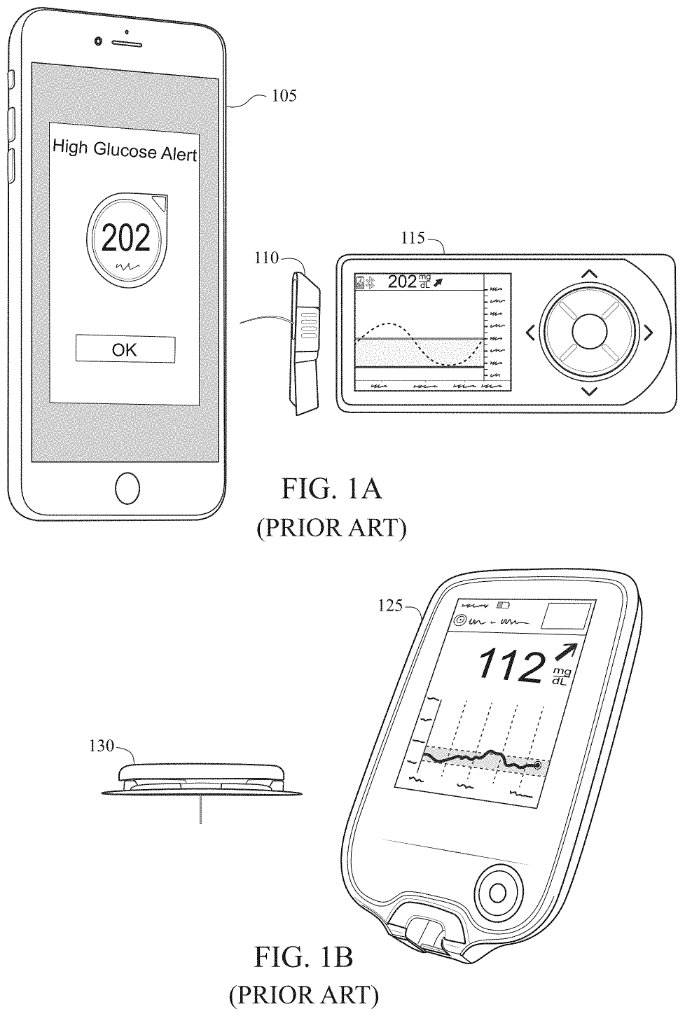

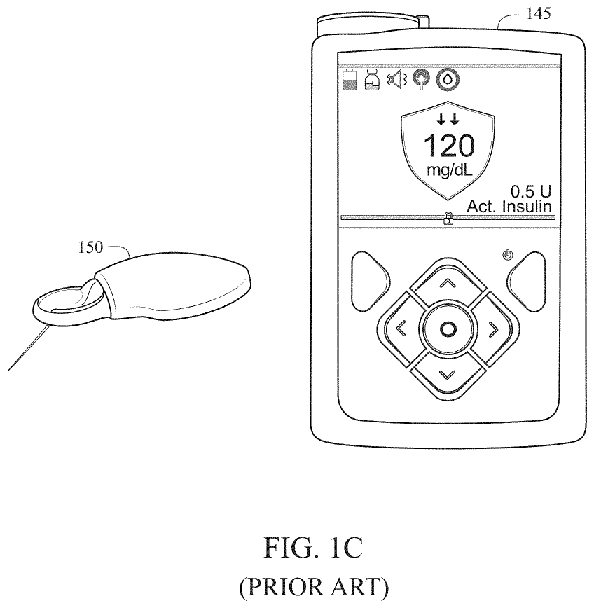

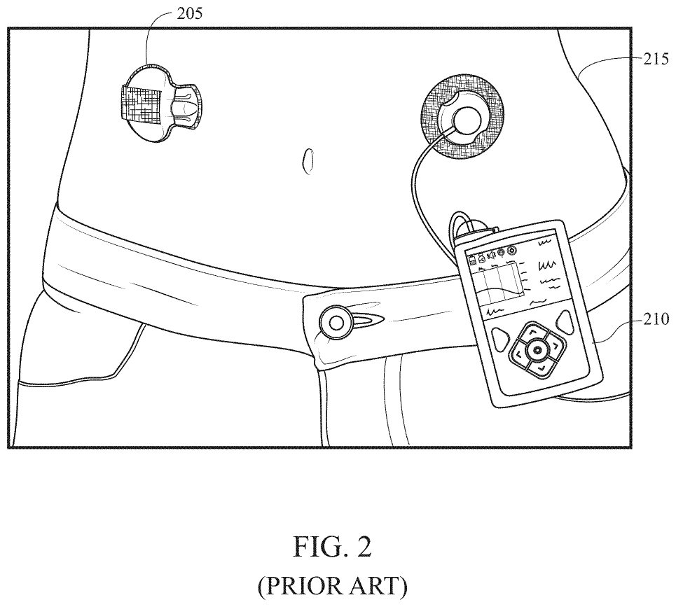

### DETAILED DESCRIPTION OF THE INVENTION

The technology disclosed herein juxtaposes the analyte sensor system and therapeutic delivery system to operate in different physiological compartments yet maintain minimum spatial separation between the two. This is achieved by dispensing the analyte sensor in the viable epidermis or dermis of a wearer, whereby the system is configured to quantify an analyte, or plurality of analytes, residing therein. Conversely, the therapeutic delivery system is dispensed in the subcutaneous region. Transverse separation of both the sensing and delivery modalities, confining the sensing routine to the viable epidermis or dermis and delivery routine to the subcutaneous adipose tissue, enables the isolation of both routines, thus mitigating likely occurrences of cross-talk, interference, contamination, and localized dilution of the analyte undergoing detection should both be co-located in a given physiological compartment. In preferential embodiments of this invention, the system functions under an open-loop paradigm whereby therapy is instigated by a user and guided by measurements from said sensor. Alternatively, the system includes a control algorithm to autonomously deliver a therapeutic intervention in response to a sensor reading or plurality of readings. It is expected that this paradigm will have profound implications for diabetes management and, in particular, those who are undergoing intensive insulin therapy.

The invention discloses simplified methods for mechanical coupling and concomitant decoupling of the analyte-selective sensor and therapeutic delivery modalities while facilitating electromagnetic interface (for the purpose of information and/or power transfer) between the same. In this manner, the therapeutic delivery contingent (i.e. insulin infusion system) can be removed and replaced while the analyte-selective sensor contingent continues unabated operation.

Body-worn analyte-selective sensors (such as continuous glucose monitors) are sensitive electrochemical systems that are configured to sense an analyte, or plurality of analytes, in a selective fashion with a high-degree of accuracy. Likewise, body-worn therapeutic delivery mechanisms (such as insulin infusion systems) are configured to deliver, in a controlled fashion, a therapeutic intervention in response to the circulating level of the analyte or plurality of analytes. In the present invention, the analyte sensor and infusion system are configured to mechanically and electromagnetically couple to one another to create a singular body-worn device comprised of these two extricable modalities.

The mechanical coupling constitutes a mechanical retention mechanism and takes the form of an interference fit, clearance fit, or transition fit, among others, to position both sensing and delivery modalities in an immutable position with respect to one another. The user engages the coupling to retain both modalities and, similarly, disengages the coupling to extricate both modalities. In alternative embodiments, a third “intermediary” element is employed to position and retain both analyte sensor and therapeutic delivery components in a fixed and immutable position. Under these embodiments, the user is still able to mechanically couple and de-couple the analyte sensing and therapeutic delivery modalities at their desire.

In a first embodiment, the user applies the analyte-selective sensor to the skin first, allowing the sensing element to penetrate the stratum corneum to access the viable epidermis or dermis to conduct the analyte sensing operation. In one particular embodiment, the sensor device consists of the analyte-selective sensor mounted on an electrical circuit board with a lock-and-key feature to allow for proper spatial orientation when mated to the therapeutic delivery mechanism such as a patch pump or other insulin infusion system. The top of the sensor device may also feature electrical contact pads or connector for mounting to the bottom of the infusion system, thereby providing a path for electrical transmission of an electromagnetic quantity to enable power or signal transfer. A sensor device mounted in this way onto the bottom of a therapeutic delivery system utilizes the embedded electrical sub-systems in the therapeutic delivery system, thereby resulting in an extremely low cost of goods for the sensor element.

Subsequently, the user applies the therapeutic delivery mechanism to the skin, the act of which causes a cannula or needle to penetrate the three upper layers of the skin (stratum corneum, epidermis and dermis) to access the subcutaneous adipose tissue, such that the therapeutic delivery mechanism mechanically couples with the analyte-selective sensor, thereby forming a singular immutable body-worn responsive therapeutic system.

In a second embodiment, the user applies the therapeutic delivery mechanism to the skin first, allowing the delivery element to penetrate the outer layers of the skin to access the subcutaneous adipose tissue to conduct the therapeutic delivery operation. Subsequently, the user applies the analyte-selective sensor to the skin, the act of which causes a microneedle or microneedle array to penetrate the stratum corneum to access the viable epidermis or dermis, such that the analyte-selective sensor mechanically couples with the therapeutic delivery mechanism, thereby forming a singular immutable body-worn responsive therapeutic system.

In a third embodiment, the user mechanically couples the analyte-selective sensor and therapeutic delivery mechanism by means of a third element—an intermediary—prior to application to the body. The intermediary retains the analyte sensor and delivery means in a fixed position with respect to one another to form a singular, body-worn responsive therapeutic system. It should be noted that the analyte-selective sensor and therapeutic system each preferably feature a skin-facing adhesive to adhere these devices in a fixed position on the surface of the wearer's body. The singular body worn device also preferably comprises a skin patch, a dermal patch, an adhesive patch, an infusion set, a patch pump, a responsive therapeutic system, or an automated therapeutic delivery system.

The electromagnetic coupling constitutes a means to convey or transfer at least one of information and power between the analyte-selective sensor and therapeutic delivery mechanism. The conveyance or transfer is either unidirectional (analyte-selective sensor to therapeutic delivery mechanism or therapeutic delivery mechanism to analyte-selective sensor) or bidirectional in nature (analyte-selective sensor to and from therapeutic delivery mechanism). The information or power transfer is either achieved wirelessly via electromagnetic waves propagating through free space or facilitated by means of an electrical connector featuring at least two conductive pads. In one scenario, the act of mechanically coupling both the analyte-selective sensor and therapeutic delivery mechanism instigates the exchange of at least one of electromagnetic information and energy between the two modalities. The use of a wireless transmission is by means of BLUETOOTH, Wi-Fi, NFC, RFID, cellular radio, ZigBee, Thread, ANT, a proprietary radio technology, a proprietary microwave technology, a proprietary millimeter-wave technology, inductive coupling, capacitive coupling, resonant coupling, or light waves.

In another embodiment, both the analyte-selective sensor and therapeutic delivery mechanism must be paired by an action of a user. It should be noted that the therapeutic delivery modality is, oftentimes, larger in spatial extent than the analyte sensing contingent, thus is better positioned to house a battery with larger charge storage capacity as well as a microelectronic system of greater level of sophistication (i.e. computational power, wireless capabilities). Accordingly, preferred embodiments include the housing of the energy source, micro-processor, and wireless data transmission capabilities within the therapeutic delivery mechanism and, optionally, the analog front end responsible for operating the analyte-selective sensor.

Another embodiment places the analog front end within the analyte-selective sensor with the remainder of electronic components (energy source, micro-processor, and wireless data transceiver) housed within the therapeutic delivery mechanism.

Another embodiment places the analog front end and the micro-processor within the analyte-selective sensor with the remainder of electronic components (energy source and wireless data transceiver) housed within the therapeutic delivery mechanism.

Another embodiment places the analog front end, the micro-processor, and energy source within the analyte-selective sensor with the wireless data transceiver housed within the therapeutic delivery mechanism.

Yet another embodiment places all electronic components (energy source, micro-processor, wireless data transceiver, and analog front end) within the analyte selective sensor such that it can operate as an extricable device in the absence of the therapeutic delivery mechanism.

Yet another embodiment of the disclosed invention includes an analyte-selective sensor positioned on the skin and configured for beyond three days of analyte monitoring (such as 7, 10, or 14 days, as in current continuous glucose monitors), whereas the therapeutic delivery mechanism is configured to be replaced every three days (such as is the case with insulin infusion systems) such that removal of the said therapeutic delivery mechanism does not require the removal of, or otherwise perturb, the analyte-selective sensor.

The analyte-selective sensor is preferably a microneedle array-based electrochemical, electrooptical, or fully electronic device configured to measure an endogenous or exogenous biochemical agent, metabolite, drug, pharmacologic, biological, or medicament in the dermal interstitium, indicative of a particular physiological or metabolic state in a physiological fluid of a user. Specifically, the microneedle array contains a plurality of microneedles, possessing vertical extent between 200 and 2000 μm, configured to selectively quantify the levels of at least one analyte located within the viable epidermis or dermis. The analyte-selective sensor is preferably an electrochemical sensor, a chemical sensor, an electrical sensor, a potentiometric sensor, an amperometric sensor, a voltammetric sensor, a galvanometric sensor, an impedimetric sensor, a conductometric sensor, or a biosensor.

The infusion system or therapeutic delivery mechanism is preferably a fluid delivery apparatus configured to provide infusion of a solution-phase therapeutic agent into the dermal interstitium, subcutaneous adipose layer, circulatory system (venous, arterial, or capillary), or musculature via a microneedle, macroneedle, hypodermic needle, cannula, catheter, or oral delivery route. The solution-phase therapeutic agent is delivered, in a controlled fashion, in response to metabolic state provided by said analyte-selective sensor.

The therapeutic agent (also referred to as “therapy”) is preferably a solution-phase drug, pharmacologic, biological, or medicament.

Yet another embodiment is a coupled system integrating a sensor and an infusion system (containing an embedded therapy) with a responsive therapeutic system. This embodiment is a body-worn device incorporating both the sensor and the infusion system to instigate the delivery of therapy, which occupies a physically-distinct compartment. Alternatively, the body-worn device incorporates the sensor, the infusion system and the therapy in a singular enclosure.

A mechanical coupling mechanism is a mechanism designed to retain the sensor within the infusion system housing or vice versa. Alternatively, the body-worn device can feature a third element (intermediary) configured to retain both sensor and infusion system within. The coupling mechanism can take the form of an interference fit, clearance fit, or transition fit.

An electromagnetic energy conveyance mechanism is an electromagnetic mechanism designed to transfer information and/or power either unidirectionally from sensor to infusion system or bidirectionally between the sensor and the infusion system. Alternatively, an intermediary, such as a third device, can effectuate the transaction between said sensor and infusion system. The conveyance mechanism can take the form of a wireless electromagnetic transmission or electromagnetic communication facilitated by a connector device.

In another embodiment, which is a closed loop embodiment, the elements are as follows:

A microneedle array analyte-selective sensor is a plurality of microneedles, possessing vertical extent between 200 and 2000 μm, configured to selectively quantify the levels of at least one analyte located within the dermal interstitium.

A therapeutic delivery mechanism, infusion system, is a fluid delivery apparatus configured to provide infusion of a solution-phase therapeutic agent into the dermal interstitium, subcutaneous adipose layer, circulatory system (venous, arterial, or capillary), or musculature via preferably a microneedle, macroneedle, hypodermic needle, cannula, catheter, or oral delivery route.

A therapeutic agent, therapy, is a solution-phase drug, pharmacologic, biological, or medicament.

A coupled system integrating sensor and infusion system (containing embedded therapy) with the responsive therapeutic system, is a body-worn device capable of incorporating both sensor and infusion system to instigate delivery of therapy occupying a physically-distinct anatomical or physiological compartment. Alternatively, the body-worn device incorporates the sensor, the infusion system, and the therapy in a singular enclosure.

A control algorithm is a software routine employing one or more mathematical transformations to control dosing of a therapeutic agent, either by means of controlling the quantity delivered, duration of delivery, and/or frequency of delivery, based on input from a user or from measurements recorded by a microneedle array analyte-selective sensor. The said dosing can either comprise bolus delivery, wherein the therapy is delivered at once, or basal delivery, wherein the therapy is delivered over an extended period of time. The mathematical transformation can employ additional inputs, either provided by a user or integrated autonomously from elsewhere.

A mechanical coupling mechanism is a mechanism designed to retain the sensor within the infusion system housing or vice versa. Alternatively, the body-worn device includes a third element (intermediary) configured to retain both the sensor and the infusion system within. The coupling mechanism preferably takes the form of an interference fit, a clearance fit, or a transition fit.

An information conveyance mechanism is an electromagnetic mechanism designed to transfer information and/or power either unidirectionally from the sensor to the infusion system or bidirectionally between the sensor and the infusion system. Alternatively, an intermediary, such as a third device, mitigates or arbitrates the transaction between the sensor and the infusion system. The conveyance mechanism preferably takes the form of a wireless electromagnetic transmission or electromagnetic communication facilitated by a connector device.

Another embodiment is an analyte-selective sensor first method embodiment. In the first step, the user applies a sensor to the skin. The analyte-selective sensor is configured to penetrate the stratum corneum to access the viable epidermis or dermis and measure the presence of an analyte or plurality of analytes in a selective fashion. In one sub-embodiment, the sensor is configured with a geometric feature that exactly matches a similar geometrical shape on the bottom of the infusion system such that the infusion system fits properly onto the sensor and makes appropriate electrical connections, if required, for the proper operation of the combined system.

In the next step, the user applies the infusion system to the skin while simultaneously engaging the infusion system to the sensor by a mechanical coupling, thereby forming a singular body-worn device. The therapeutic delivery transducer is configured to penetrate the stratum corneum and deliver, in a controlled fashion, a solution-phase therapeutic agent or collection of therapeutic agents to a physiological compartment beneath the stratum corneum, the positioning requiring a mechanical retention between the therapeutic delivery mechanism and the analyte-selective sensor to form a singular body-worn device.

In the next step, the conveyance of relevant information and/or power between the sensor and the infusion system occurs. An electromagnetic signal transferring power and/or carrying information relevant to the sensor and/or the infusion system is relayed between the two contingents, either in a unidirectional fashion (sensor to infusion system/infusion system to sensor) or bidirectional fashion (sensor to and from infusion system) or via an intermediary (sensor to intermediary to infusion system/infusion system to intermediary to sensor/sensor to and from intermediary to and from infusion system).

Another embodiment is therapeutic delivery mechanism first method embodiment. At a first step, a user applies an infusion system to the skin. The therapeutic delivery mechanism is configured to penetrate the stratum corneum and deliver, in a controlled fashion, a solution-phase therapeutic agent or collection of therapeutic agents to a physiological compartment beneath the stratum corneum.

Next, the user applies the sensor to the skin while simultaneously engaging the sensor to the infusion system by a mechanical coupling, thereby forming a singular body-worn device. The analyte-selective sensor is configured to penetrate the stratum corneum to access the viable epidermis or dermis and measure the presence of an analyte or plurality of analytes in a selective fashion, the positioning requiring a mechanical retention between the analyte-selective sensor and the therapeutic delivery mechanism to form a singular body-worn device. The dermis preferably includes the dermal interstitium, the dermal interstitial fluid, the papillary dermis, the reticular dermis, or the dermal capillary bed. The analyte or plurality of analytes preferably includes at least one of glucose, lactate, a ketone body, uric acid, ascorbic acid, alcohol, glutathione, hydrogen peroxide, a metabolite, an electrolyte, an ion, a drug, a pharmacologic, a biological, or a medicament.

Next, the conveyance of relevant information between the sensor and the infusion system occurs. An electromagnetic signal transferring power and/or carrying information relevant to the sensor and/or the infusion system is relayed between the two contingents, either in a unidirectional fashion (sensor to infusion system/infusion system to sensor) or bidirectional fashion (sensor to and from infusion system) or via an intermediary (sensor to intermediary to infusion system/infusion system to intermediary to sensor/sensor to and from intermediary to and from infusion system).

Another embodiment is a coupling of analyte selective sensor and a therapeutic delivery mechanism method. First, a sensor engages an infusion system by a mechanical coupling. This is the mechanical coupling of analyte-selective sensor and therapeutic delivery mechanism to form a singular device primed to be applied to the skin. Optionally, a third element is employed to retain both the analyte-selective sensor and therapeutic delivery mechanism prior to application to the skin.

Next, the user applies the coupled sensor and infusion system as a singular device to the skin. Both the analyte-selective sensor and the therapeutic delivery mechanism, comprising a singular mechanically coupled device, are applied simultaneously to the skin. The analyte-selective sensor is configured to penetrate the stratum corneum to access the viable epidermis or dermis and measure the presence of an analyte or plurality of analytes in a selective fashion. The therapeutic delivery mechanism is configured to penetrate the stratum corneum and deliver, in a controlled fashion, a solution-phase therapeutic agent or collection of therapeutic agents to a physiological compartment beneath the stratum corneum.

Next, the conveyance of relevant information between the sensor and the infusion system occurs. An electromagnetic signal transferring power and/or carrying information relevant to the sensor and/or the infusion system is relayed between the two contingents, either in a unidirectional fashion (sensor infusion system/infusion system sensor) or bidirectional fashion (sensor infusion system) or via an intermediary (sensor intermediary infusion system/sensor intermediary infusion system).

The inputs of the invention are the coupling, which is a mechanism designed to retain the sensor within the infusion system housing or vice versa. Alternatively, the body-worn device includes a third element configured to retain both the sensor and the infusion system within. The coupling mechanism preferably takes the form of an interference fit, clearance fit, or transition fit. The outputs of the invention are the conveyance which is an electromagnetic signal carrying power and/or information relevant to the sensor and/or the infusion system is relayed between the two contingents, either in a unidirectional fashion (sensor to infusion system/infusion system to sensor) or bidirectional fashion (sensor to and from infusion system) or via an intermediary (sensor to intermediary to infusion system/sensor to and from intermediary to and from infusion system).

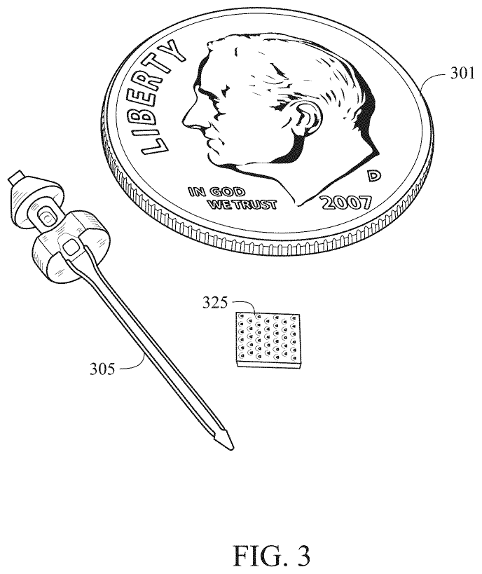

A sensor is preferably a plurality of microneedles, possessing vertical extent between 200 and 2000 μm, configured to selectively quantify the levels of at least one analyte located within the viable epidermis or dermis. FIG. 3 illustrates the microneedle array sensor 325 in relation to a dime 301 and needle 305.

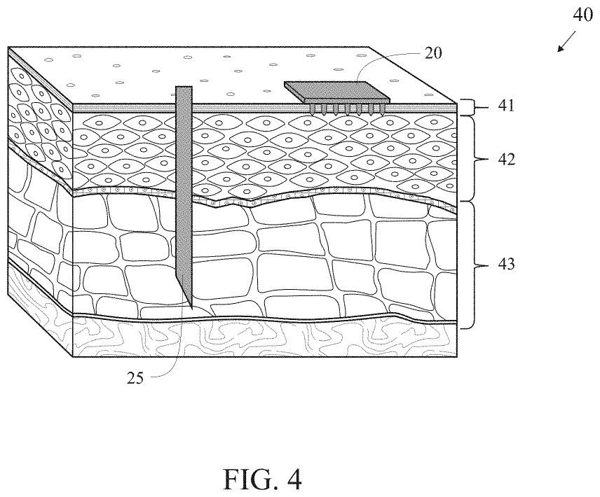

FIG. 4 is a pictorial representation 40 of a therapeutic delivery device 25 configured to operate within the subcutaneous tissue 43 and an analyte-selective sensor 20 configured to operate within the dermis 42, and through the epidermis 41. It should be noted that both are located in close spatial proximity.

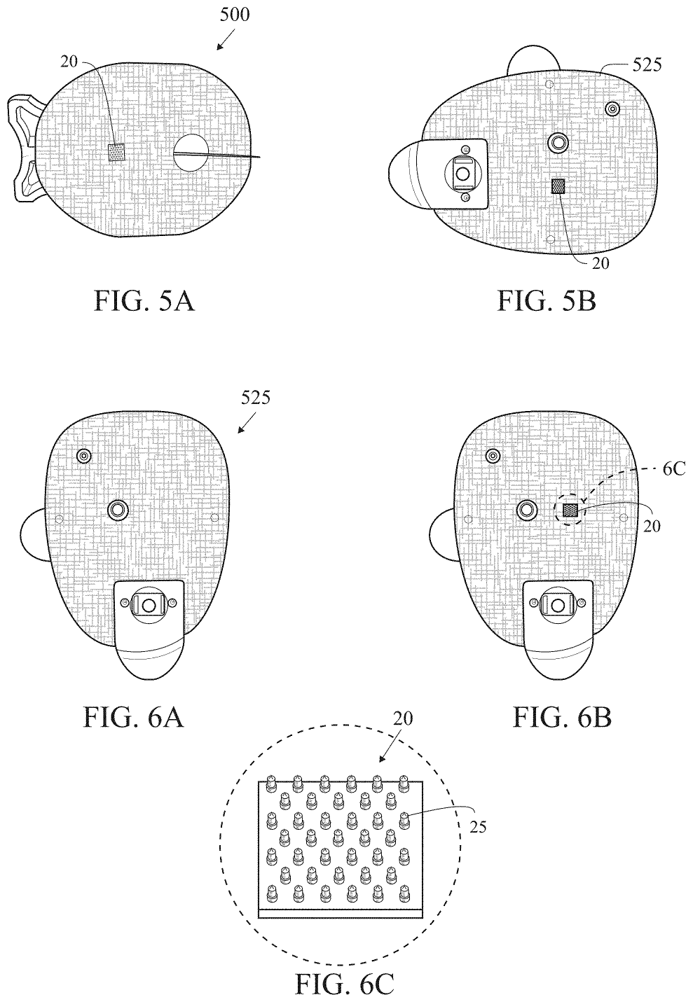

FIG. 5A is an illustration of an integration of a microneedle array-based analyte-selective sensor 20 into an infusion set 500.

FIGS. 5B, 6A and 6B illustrate the integration of a microneedle array-based analyte-selective sensor 20 into a patch pump 525. FIG. 6C illustrates the microneedle array-based analyte-selective sensor 20 and microneedles 25.

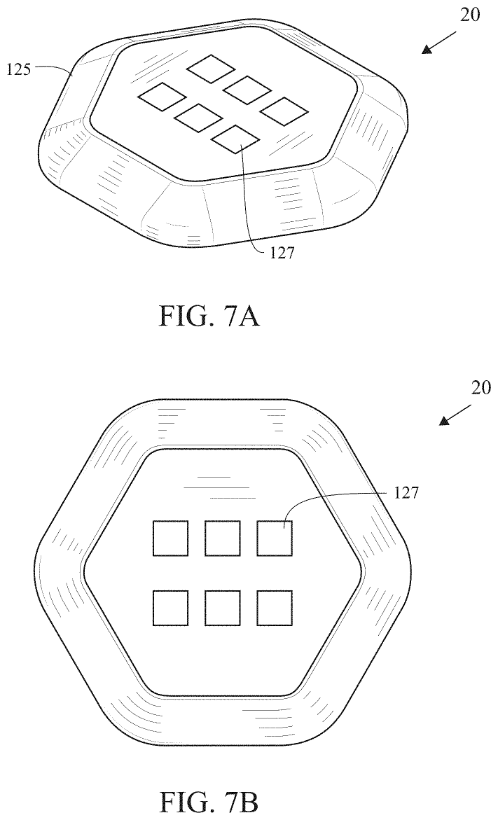

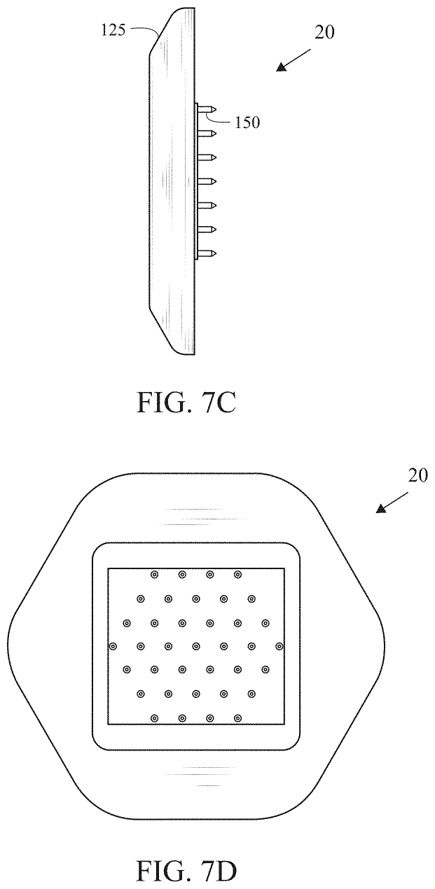

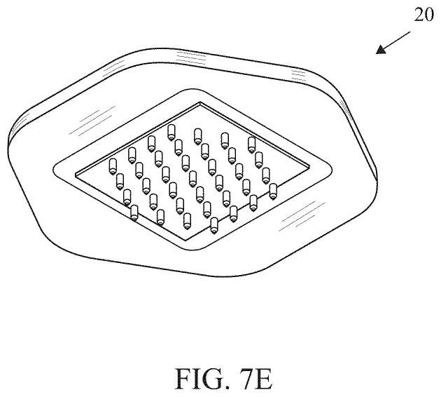

FIGS. 7A, 7B, 7C, 7D and 7E illustrate a microneedle-based analyte-selective sensor 20 with a microneedle array and excitation and measurement circuit, not shown. The microneedle analyte-selective sensor 20 preferably comprises a housing member 125, a back plate, an internal pad, a circuit board cover, an external pad, and adhesive pad, a front panel, microneedles 150, and a printed circuit board containing the electronic circuitry required to transduce biochemical signals to digital data that are wirelessly transmitted to an external device via the embedded wireless transceiver. An electrochemical analog front end preferably includes: a Texas Instruments LMP91000 Sensor AFE System, configurable APE potentiostat for low-power chemical sensing applications; a Texas Instruments LMP91200 configurable AFE for low-power chemical sensing applications; or an Analog Devices ADuCM350 16-Bit Precision, low power meter on a chip with Cortex-M3 and connectivity. The wireless transceiver is preferably is a BLUEGIGA BLE-113A BLUETOOTH Smart Module, or a Texas Instruments CC2540 SimpleLink BLUETOOTH Smart Wireless MCU with USB. The micro-needles 150 penetrate the stratum corneum to access the viable epidermis or dermis. The microneedle-based analyte-selective sensor 20 also has multiple electrical pads 127 for transferring power and/or information between the therapeutic delivery system and microneedle-based analyte-selective sensor 20. The microneedle-based analyte-selective sensor 20 preferably has between two and ten pads 127, and most preferably six pads 127.

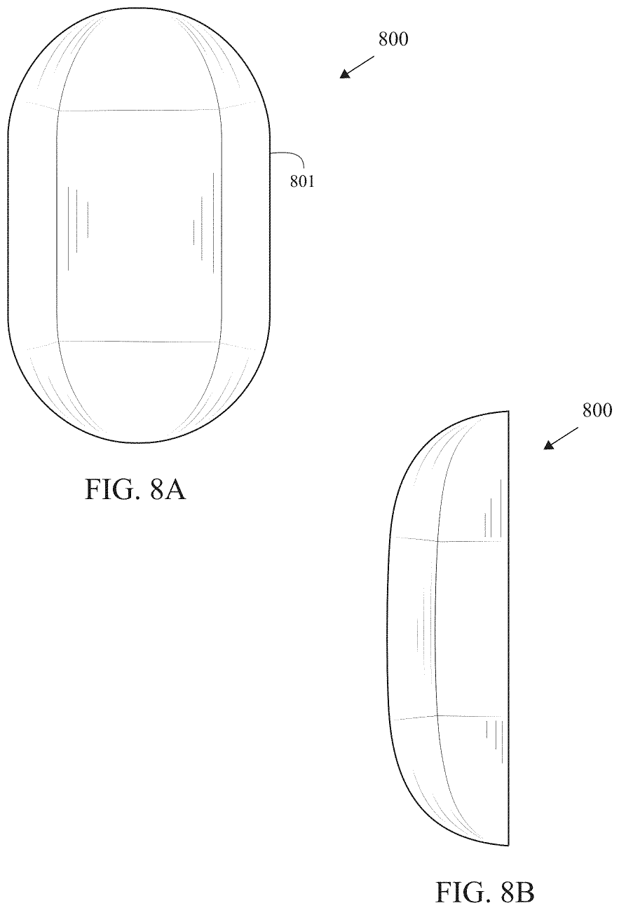

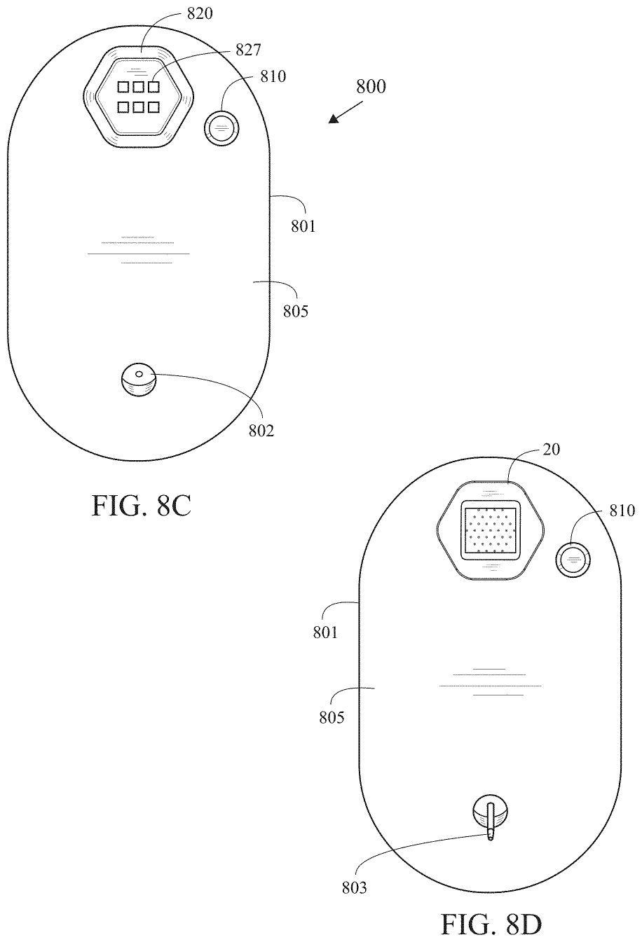

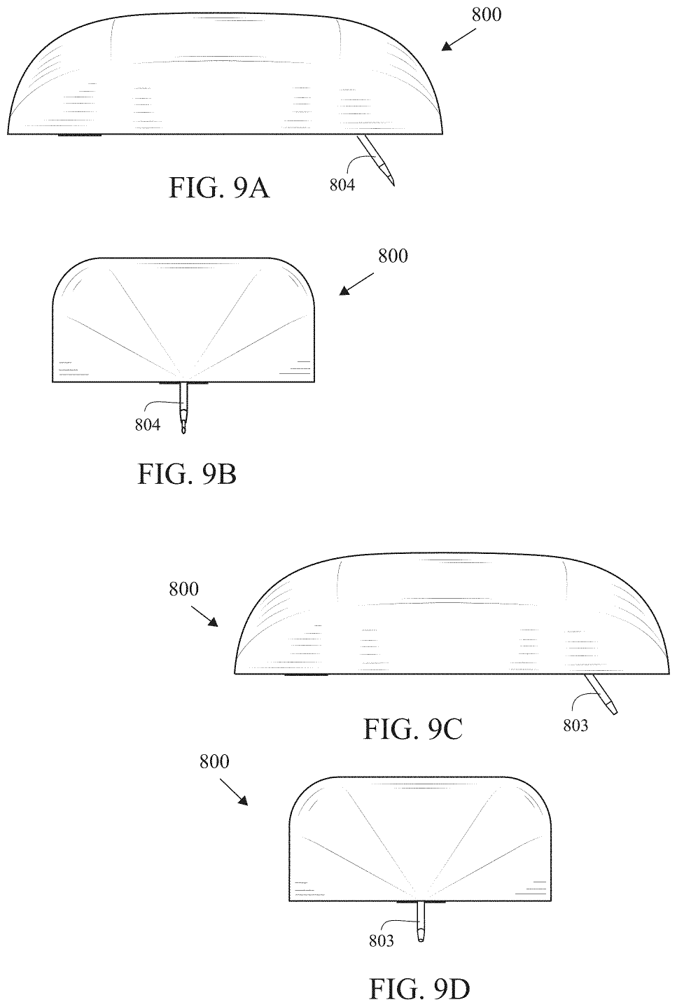

FIGS. 8A-8C illustrate a therapeutic delivery system 800 having a body 801 with an internal surface 805 with a port 802, a button 810, cavity 820 having multiple electrical pads 827 for coupling with microneedle-based analyte-selective sensor 20. FIG. 8D illustrates the therapeutic delivery system 800 coupled with the microneedle-based analyte-selective sensor 20 and a cannula 803 attached to the port 802. FIGS. 9A and 9B illustrate the therapeutic delivery system 800 with a needle 804. FIGS. 9C and 9D illustrate the therapeutic delivery system 800 with a cannula 803 attached to the port 802.

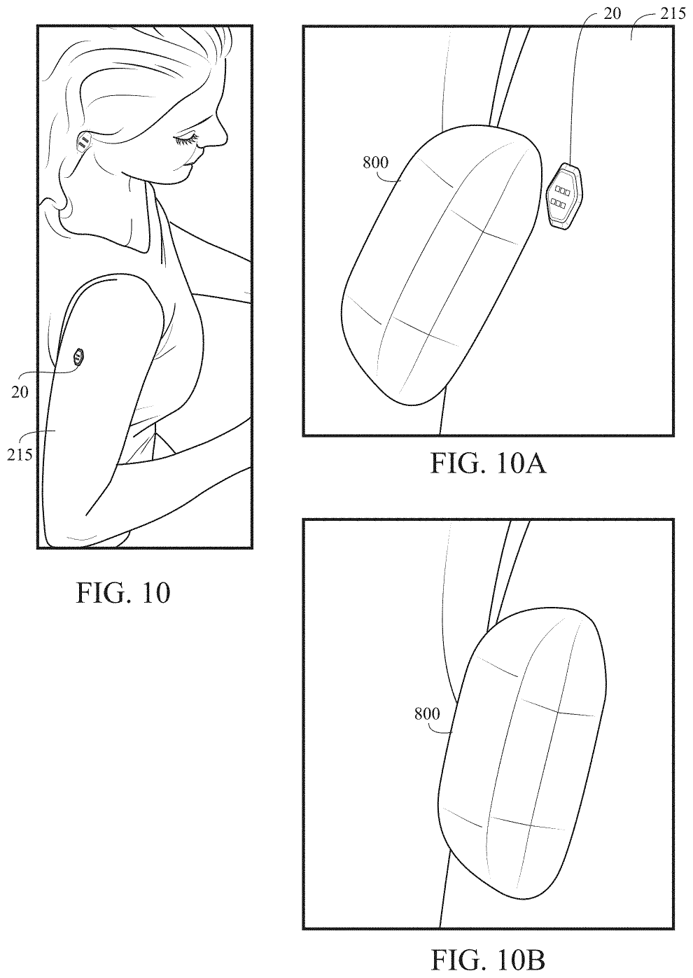

FIGS. 10, 10A and 10B are an illustration of an application process of the analyte-selective sensor and therapeutic delivery system, illustrative of the mechanical and electromagnetic coupling operation between the same. In FIG. 10, the application of the analyte-selective sensor to the skin of a wearer 215 is shown, with the analyte-selective sensor 20 featuring six conductive electrical pads 127 for conveyance of electromagnetic energy (power and/or information). In FIG. 10A, the application of the therapeutic delivery system 800 on the posterior of the analyte-selective sensor is shown. In FIG. 10B, the mechanically- and electromagnetically-coupled analyte-selective sensor 20 and therapeutic delivery system 800 on the skin of a wearer 215 is shown.

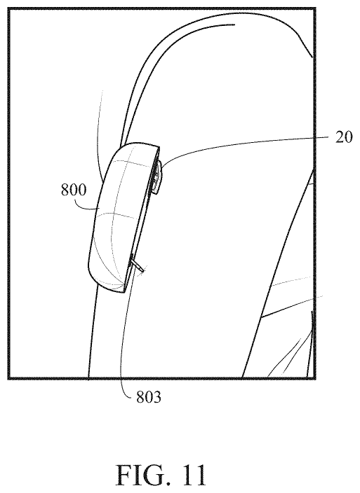

FIG. 11 is an illustration of the mechanically- and electromagnetically-coupled analyte-selective sensor 20 and therapeutic delivery system 800 with the microneedle array of the analyte-selective sensor 20 and the cannula 803 of the therapeutic delivery system 20 shown.

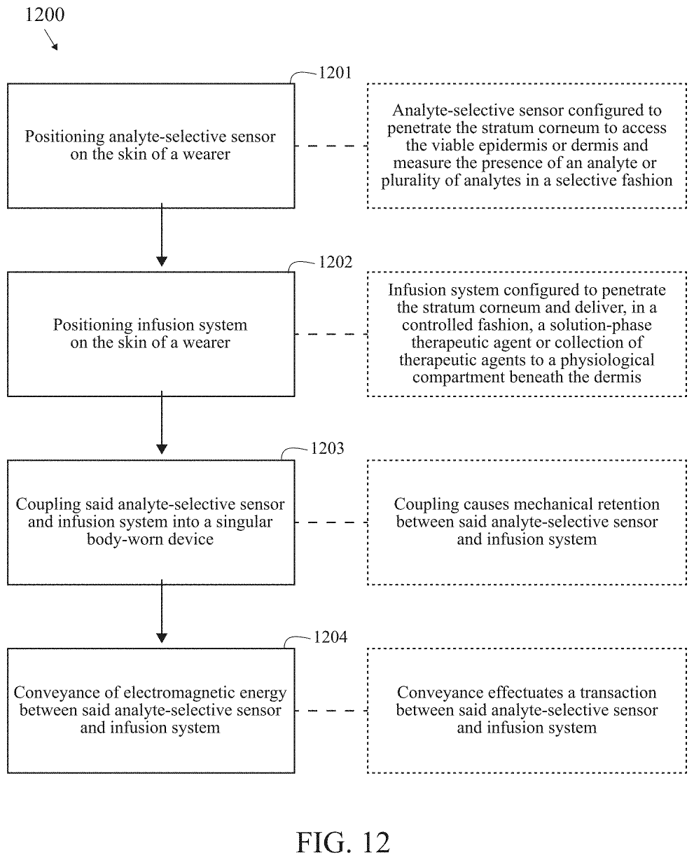

FIG. 12 illustrates a method 1200 for coupling of an analyte-selective sensor and an infusion system into a singular body-worn device. At block 1201, an analyte-selective sensor is positioned on the skin of a wear. The analyte-selective sensor is configured to penetrate the stratum corneum to access the viable epidermis or dermis and measure the presence of an analyte or plurality of analytes in a selective fashion. At block 1202, the infusion system is positioned on the skin of a wearer. The infusion system is configured to penetrate the stratum corneum and deliver, in a controlled fashion, a solution-phase therapeutic agent or collection of therapeutic agents to a physiological compartment beneath the dermis. At block 1203, the analyte-selective sensor is coupled to the infusion system to form a singular body-worn device. The coupling causes a mechanical retention between the analyte-selective sensor and the infusion system. At block 1204, electromagnetic energy is conveyed between the analyte-selective sensor and the infusion system. The conveyance effectuates a transaction between the analyte-selective sensor and the infusion system.

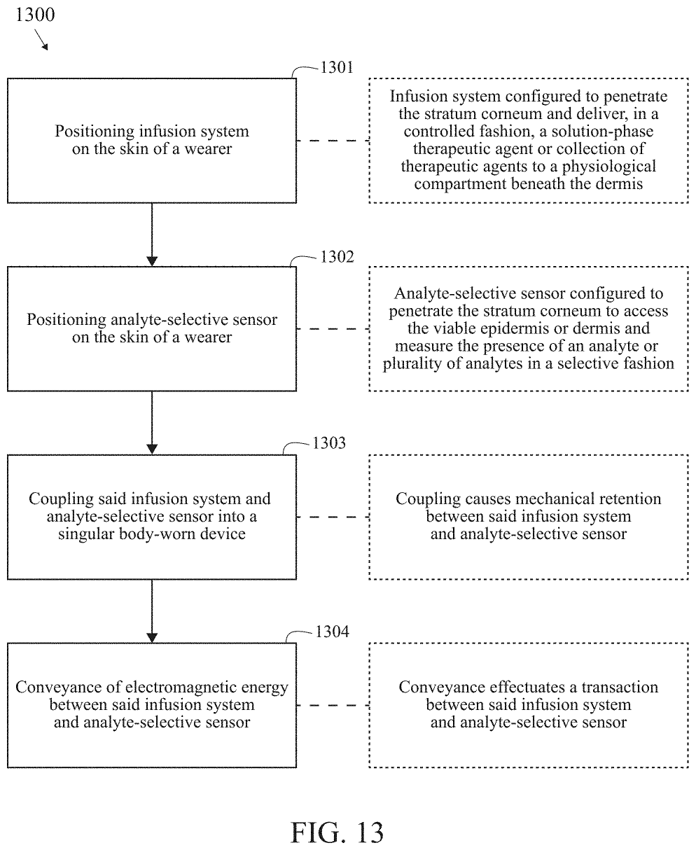

FIG. 13 illustrates a method 1300 for coupling of an infusion system and an analyte-selective sensor into a singular body-worn device. At block 1301, the infusion system is positioned on the skin of a wearer. The infusion system is configured to penetrate the stratum corneum and deliver, in a controlled fashion, a solution-phase therapeutic agent or collection of therapeutic agents to a physiological compartment beneath the dermis. At block 1302, an analyte-selective sensor is positioned on the skin of a wear. The analyte-selective sensor is configured to penetrate the stratum corneum to access the viable epidermis or dermis and measure the presence of an analyte or plurality of analytes in a selective fashion. At block 1303, the analyte-selective sensor is coupled to the infusion system to form a singular body-worn device. The coupling causes a mechanical retention between the analyte-selective sensor and the infusion system. At block 1304, electromagnetic energy is conveyed between the analyte-selective sensor and the infusion system. The conveyance effectuates a transaction between the analyte-selective sensor and the infusion system.

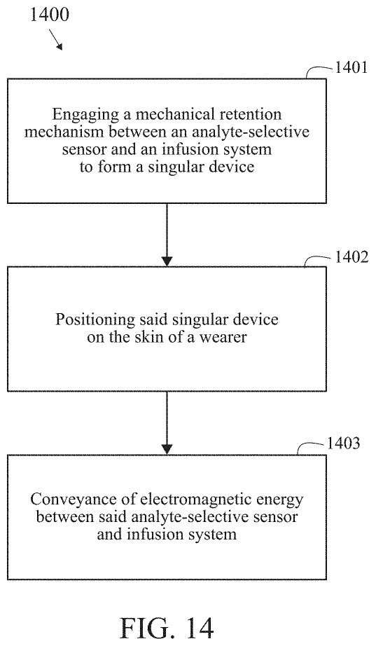

FIG. 14 illustrates a flow chart for a method 1400 for the analyte-selective sensor and infusion system coupling prior to skin application. At block 1401, a mechanical retention mechanism engages between an analyte-selective sensor and an infusion system to form a singular body-worn device. At block 1402, the singular body-worn device is positioned on the skin of a wearer. At block 1403, electromagnetic energy is conveyed between the analyte-selective sensor and the infusion system.

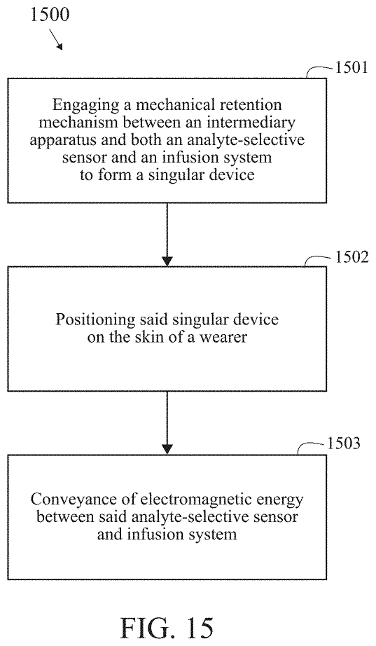

FIG. 15 illustrates a flow chart for a method 1500 for the analyte-selective sensor and infusion system coupling, facilitated by an intermediary apparatus, prior to skin application. At block 1501, a mechanical retention mechanism engages between an intermediary device and both an analyte-selective sensor and an infusion system to form a singular body-worn device. At block 1502, the singular body-worn device is positioned on the skin of a wearer. At block 1503, electromagnetic energy is conveyed between the analyte-selective sensor and the infusion system.

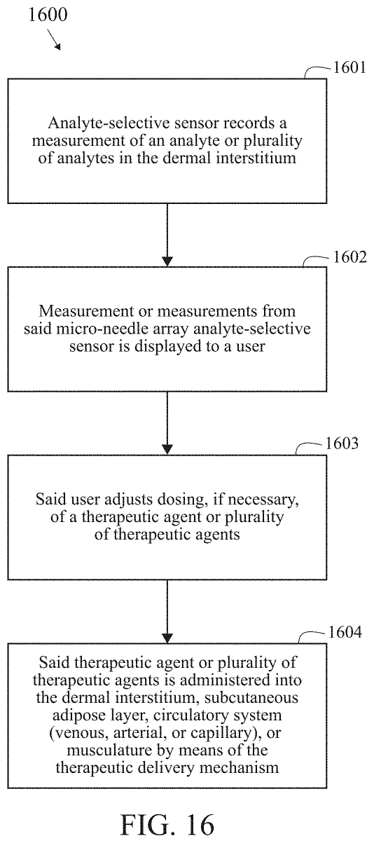

FIG. 16 is a flow chart for a method 1600 of an open loop embodiment of the invention. The method 1600 for performing the open loop embodiment begins at block 1601 with a microneedle array analyte-selective sensor recording a measurement of an analyte or plurality of analytes in the dermal interstitium. Circulating levels of an analyte within the viable epidermis or dermis is quantified by means of the sensor. Next, at block 1602, a measurement or measurements from the microneedle array analyte-selective sensor is displayed to a user. The user receives a reading of the circulating level of an analyte or plurality of analytes on a display or interface. Alternatively, user receives notification that the circulating level of an analyte or plurality of analytes extends beyond a pre-defined criteria or range of values. Next, at block 1603, the user adjusts dosing, if necessary, of a therapeutic agent or plurality of therapeutic agents. The user manipulates a quantity, duration, or frequency of infusion of the therapy based on measurement of analyte or plurality of analytes tendered by the sensor. Next, at block 1604, the therapeutic agent or plurality of therapeutic agents is administered into the dermal interstitium, subcutaneous adipose layer, circulatory system (venous, arterial, or capillary), or musculature by means of the therapeutic delivery mechanism. The therapy is delivered to the user via the infusion sub-system and is based on the user's determination of dosage given measurement or measurements from the sensor.

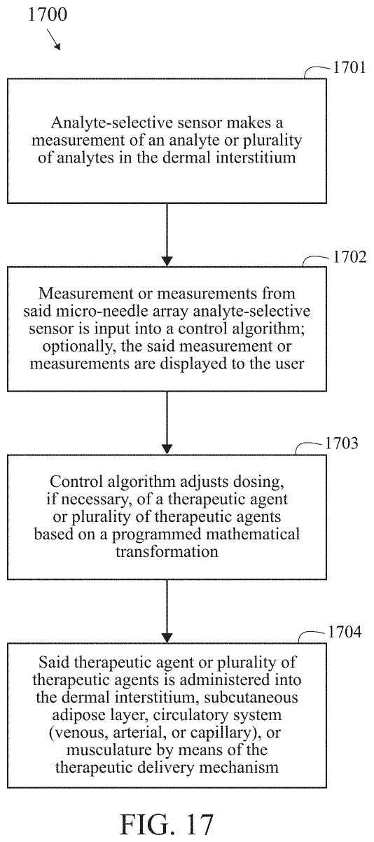

FIG. 17 is a flow chart for a method 1700 of a closed loop embodiment of the invention. The method 1700 for performing the closed loop embodiment begins at block 1701 with a microneedle array analyte-selective sensor recording a measurement of an analyte or plurality of analytes in the dermal interstitium. Circulating levels of an analyte within the viable epidermis or dermis is quantified by means of the sensor. Next, at block 1702, a measurement or measurements from the microneedle array analyte-selective sensor is input into a control algorithm; optionally, the measurement or measurements are displayed to the user. Current and, optionally, past stored measurements are employed as input or inputs into the algorithm. Alternatively, the user also receives a reading of the circulating level of an analyte or plurality of analytes on a display or interface. Alternatively, the user receives notification that the circulating level of an analyte or plurality of analytes extends beyond a pre-defined criteria or range of values. Next, at block 1703, the control algorithm adjusts dosing, if necessary, of a therapeutic agent or plurality of therapeutic agents based on a programmed mathematical transformation. The algorithm autonomously manipulates a quantity, duration, or frequency of infusion of the therapy based on measurement of analyte or plurality of analytes tendered by the sensor. Next, at block 1704, the therapeutic agent or plurality of therapeutic agents is administered into the dermal interstitium, subcutaneous adipose layer, circulatory system (venous, arterial, or capillary), musculature by means of the therapeutic delivery mechanism. The therapy is delivered to the user via the infusion sub-system and is based on the determination of dosage given output of the algorithm.

The input of circulating levels of an analyte or plurality of analytes within the viable epidermis or dermis is an endogenous or exogenous biochemical agent, metabolite, drug, pharmacologic, biological, or medicament in the viable epidermis or dermis, indicative of a particular physiological or metabolic state.

The output is an administration of a therapeutic agent or plurality of therapeutic agents into the circulatory system (venous, arterial, or capillary), musculature or oral delivery route. A measurement tendered by the sensor is employed to instigate the release of the therapy by means of the infusion sub-system. In the open loop embodiment, the delivery of the therapy is controlled by a user. In the closed loop embodiment, the algorithm is employed to control the dose, duration, and frequency of the therapy.

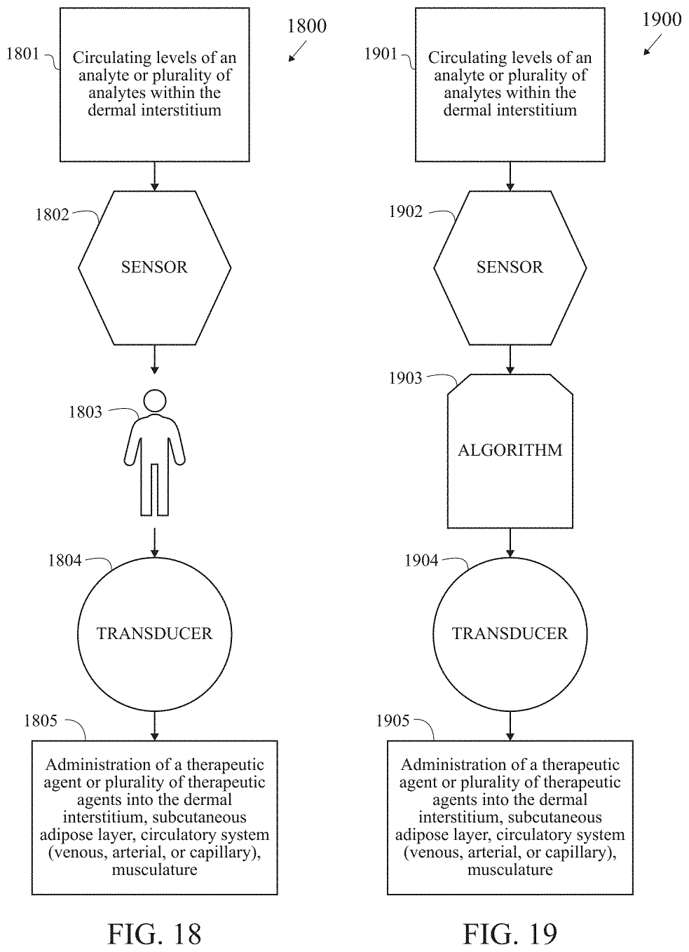

FIG. 18 is a block/process flow diagram 1800 illustrating the inputs, outputs, and major constituents under the open loop embodiment. At block 1801, circulating levels of an analyte or an analytes are within the dermis. At block 1802, a sensor measures the analytes. The user 1803 adjusts dosing, if necessary, of a therapeutic agent or plurality of therapeutic agents. The user 1803 manipulates a quantity, duration, or frequency of infusion of the therapy 1804 based on measurement of analyte or plurality of analytes tendered by the sensor. At block 1805, the therapeutic agent or plurality of therapeutic agents is administered into the dermal interstitium, the subcutaneous adipose layer, circulatory system (venous, arterial, or capillary) musculature by means of the therapeutic delivery mechanism. The therapy is preferably delivered to the user via the infusion sub-system and is based on the user's determination of dosage given measurement or measurements from the sensor.

FIG. 19 is a block/process flow diagram 1900 illustrating the inputs, outputs, and major constituents under the closed loop embodiment. At block 1901, circulating levels of an analyte or an analytes are within the dermis. At block 1902, a sensor measures the analytes. The control algorithm 1903 adjusts dosing, if necessary, of a therapeutic agent or plurality of therapeutic agents based on a programmed mathematical transformation. The algorithm autonomously manipulates a quantity, duration, or frequency of infusion of the therapy 1904 based on measurement of analyte or plurality of analytes tendered by the sensor. Next, at block 1905, the therapeutic agent or plurality of therapeutic agents is administered into the subcutaneous adipose layer, circulatory system (venous, arterial, or capillary), musculature or oral delivery route by means of the therapeutic delivery mechanism. The therapy is delivered to the user via the infusion sub-system and is based on the determination of dosage given output of the algorithm.

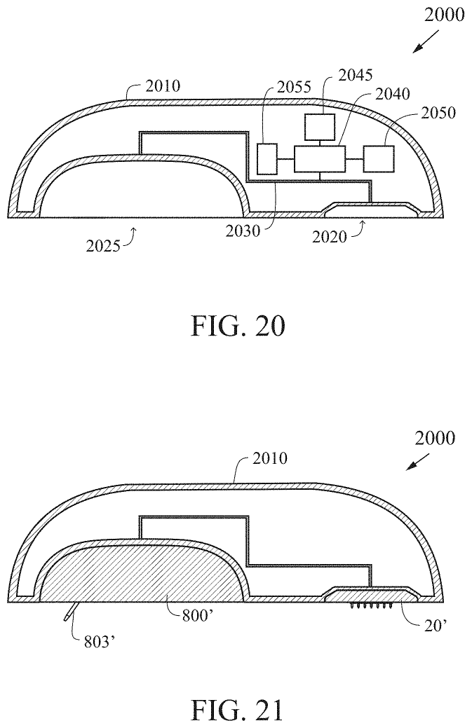

FIGS. 20 and 21 illustrate an alternative embodiment which has an intermediary device 2010 integrated with an infusion system 800′ and an analyte-selective sensor 20′ into a singular body-worn device 2000. The intermediary device 2010 includes a compartment 2025 for removable integration with an infusion system 800′, a compartment 2020 for removable integration with an analyte-selective sensor 20′, a CPU 2040, a memory 2045, a transceiver 2050, an interface 2055, and a communication/connection line 2030. In this embodiment, the user mechanically couples the analyte-selective sensor 20′ and infusion system (therapeutic delivery mechanism) 800′ by means of the intermediary device 2010, preferably prior to application to the wearer's body. The intermediary device 2010 retains the analyte sensor 20′ and infusion system 800′ in a fixed position with respect to one another to form a singular, body-worn responsive therapeutic system 2000. The singular body worn device 2000 preferably also comprises a skin patch, a dermal patch, an adhesive patch, an infusion set, a patch pump, a responsive therapeutic system, or an automated therapeutic delivery system. In alternative embodiments, connection line 2030 is not present, and the communication between the infusion system 800′ and the analyte-selective sensor 20′ is wireless. In a most preferred embodiment, the intermediary device 2010 preferably has a length ranging from 2 centimeters (cm) to 13 cm, a width ranging from 1 cm to 8 cm, and a height ranging from 1 cm to 8 cm. The analyte-selective sensor 20′ preferably has a diameter ranging from 1 cm to 5 cm and a thickness ranging from 0.1 cm to 3 cm. The infusion system preferably has a length ranging from 2 centimeters (cm) to 12 cm, a width ranging from 1 cm to 7 cm, and a height ranging from 1 cm to 7 cm.

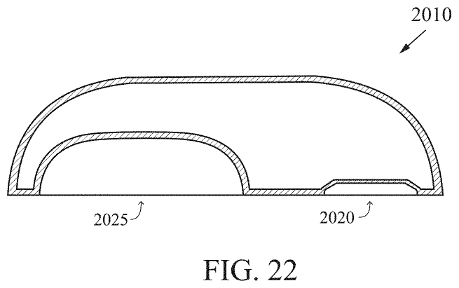

FIG. 22 illustrates an embodiment with the intermediary device 2010 as a shell for retaining in recess 2025 an infusion system and retaining in recess 2020 an analyte-selective sensor to form a singular body-worn device. In this embodiment, the intermediary device is preferably formed of plastic and contains no electrical components. The recesses 2025 and 2020 can be shaped to retain various shapes of sensors and infusion systems.

McCanna et al., U.S. Pat. No. 9,933,387 for a Miniaturized Sub-Nanoampere Sensitivity Low-Noise Potentiostat System is hereby incorporated by reference in its entirety.

Windmiller, U.S. patent application Ser. No. 15/177,289, filed on Jun. 8, 2016, for a Methods And Apparatus For Interfacing A Microneedle-Based Electrochemical Biosensor With An External Wireless Readout Device is hereby incorporated by reference in its entirety.

Wang et al., U.S. Patent Publication Number 20140336487 for a Microneedle Arrays For Biosensing And Drug Delivery is hereby incorporated by reference in its entirety.

Windmiller, U.S. patent Ser. No. 10/092,207 for a Tissue Penetrating Electrochemical Sensor Featuring A Co Electrodeposited Thin Film Comprised Of A Polymer And Bio-Recognition Element is hereby incorporated by reference in its entirety.

Windmiller, et al., U.S. patent application Ser. No. 15/913,709, filed on Mar. 6, 2018, for Methods For Achieving An Isolated Electrical Interface Between An Anterior Surface Of A Microneedle Structure And A Posterior Surface Of A Support Structure is hereby incorporated by reference in its entirety.

PCT Publication Number WO2018071265 for an Electro-Deposited Conducting Polymers For The Realization Of Solid-State Reference Electrodes For Use In Intracutaneous And Subcutaneous Analyte-selective Sensors is hereby incorporated by reference in its entirety.

Windmiller et al., U.S. patent application Ser. No. 15/961,793, filed on Apr. 24, 2018, for Heterogeneous Integration Of Silicon-Fabricated Solid Microneedle Sensors And CMOS Circuitry is hereby incorporated by reference in its entirety.

Windmiller et al., U.S. patent application Ser. No. 16/051,398, filed on Jul. 13, 2018, for Method And System For Confirmation Of Microneedle-Based Analyte-Selective Sensor Insertion Into Viable Tissue Via Electrical Interrogation is hereby incorporated by reference in its entirety.

Windmiller et al., U.S. patent application Ser. No. 16/701,784, filed on Dec. 3, 2019, for Devices And Methods For The Generation Of Alerts Due To Rising Levels Of Circulating Ketone Bodies In Physiological Fluids is hereby incorporated by reference in its entirety.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention the following:

1. A method for the coupling of an analyte-selective sensor and an infusion system into a singular body-worn device, said method comprising:
positioning said analyte-selective sensor on the skin of a wearer, said analyte-selective sensor configured to penetrate the stratum corneum to access the viable epidermis or dermis and measure the presence of an analyte or plurality of analytes in a selective fashion;
positioning said infusion system on the skin of a wearer, said infusion system configured to penetrate the stratum corneum and deliver a solution-phase therapeutic agent or collection of therapeutic agents to a physiological compartment beneath the dermis, said positioning requiring a mechanical retention between said infusion system and said analyte-selective sensor to form a singular body-worn device; and
conveying electromagnetic energy between said analyte-selective sensor and infusion system to effectuate a transaction between said analyte-selective sensor and infusion system.

2. The method of claim 1 in which the said analyte-selective sensor contains a geometric feature configured to mechanically couple with a corresponding structure on the bottom of the said infusion system during the said positioning of said infusion system on the skin of a wearer, thereby allowing the said infusion system to be placed in a proper spatial orientation on the top surface of the said analyte-selective sensor already applied to the skin of said wearer.

3. The method of claim 1, wherein said analyte-selective sensor is first positioned on the skin of the wearer.

4. The method of claim 1, wherein said analyte-selective sensor is an electrochemical sensor, a chemical sensor, an electrical sensor, a potentiometric sensor, an amperometric sensor, a voltammetric sensor, a galvanometric sensor, an impedimetric sensor, a conductometric sensor, or a biosensor.

5. The method of claim 1, wherein said infusion system comprises a fluid delivery apparatus configured to provide infusion via a microneedle, microneedle array, macroneedle, hypodermic needle, cannula, catheter, or oral delivery route.

6. The method of claim 1, wherein said singular body-worn device comprises a skin patch, a dermal patch, an adhesive patch, an infusion set, a patch pump, a responsive therapeutic system, or an automated therapeutic delivery system.

7. The method of claim 1, wherein said analyte or plurality of analytes includes at least one of glucose, lactate, a ketone body, uric acid, ascorbic acid, alcohol, glutathione, hydrogen peroxide, a metabolite, an electrolyte, an ion, a drug, a pharmacologic, a biological, or a medicament.

8. The method of claim 1, wherein said conveyance of electromagnetic energy is for the purpose of transferring at least one of information and energy.

9. The method of claim 1, wherein said conveyance of electromagnetic energy is by means of an electrical connector featuring conductive elements or by a wireless transmission.

10. A method for the coupling of an analyte-selective sensor configured to penetrate the stratum corneum to access the viable epidermis or dermis and measure the presence of an analyte or plurality of analytes in a selective fashion and an infusion system configured to penetrate the stratum corneum and deliver, in a controlled fashion, a solution-phase therapeutic agent or collection of therapeutic agents to a physiological compartment beneath the dermis, said method comprising:
engaging a mechanical retention mechanism between said analyte-selective sensor and said infusion system to form a singular device;
positioning said singular device on the skin of a wearer; and
conveying electromagnetic energy between said analyte-selective sensor and infusion system to effectuate a transaction between said analyte-selective sensor and infusion system.

11. The method of claim 10, wherein said analyte-selective sensor is an electrochemical sensor, a chemical sensor, an electrical sensor, a potentiometric sensor, an amperometric sensor, a voltammetric sensor, a galvanometric sensor, an impedimetric sensor, a conductometric sensor, or a biosensor.

12. The method of claim 10, wherein said analyte or plurality of analytes includes at least one of glucose, lactate, a ketone body, uric acid, ascorbic acid, alcohol, glutathione, hydrogen peroxide, a metabolite, an electrolyte, an ion, a drug, a pharmacologic, a biological, or a medicament.

13. The method of claim 10, wherein said infusion system comprises a fluid delivery apparatus configured to provide infusion via a microneedle, microneedle array, macroneedle, hypodermic needle, cannula, catheter, or oral delivery route.

14. The method of claim 10, wherein said singular device comprises a skin patch, a dermal patch, an adhesive patch, an infusion set, a patch pump, a responsive therapeutic system, or an automated therapeutic delivery system.

15. The method of claim 10, wherein said conveyance of electromagnetic energy is for the purpose of transferring at least one of information and energy.

16. The method of claim 10, wherein said conveyance of electromagnetic energy is by means of an electrical connector featuring conductive elements or by a wireless transmission.

17. A method for the coupling of an analyte-selective sensor configured to penetrate the stratum corneum to access the viable epidermis or dermis and measure the presence of an analyte or plurality of analytes in a selective fashion and an infusion system configured to penetrate the stratum corneum and deliver, in a controlled fashion, a solution-phase therapeutic agent or collection of therapeutic agents to a physiological compartment beneath the dermis, said method comprising:
engaging a mechanical retention mechanism between an intermediary apparatus and both said analyte-selective sensor and said infusion system to form a singular device;
positioning said singular device on the skin of a wearer; and
conveying electromagnetic energy between said analyte-selective sensor and infusion system to effectuate a transaction between said analyte-selective sensor and infusion system.

18. The method of claim 17, wherein said analyte-selective sensor is a microneedle or a microneedle array.

19. The method of claim 17, wherein said analyte or plurality of analytes includes at least one of glucose, lactate, a ketone body, uric acid, ascorbic acid, alcohol, glutathione, hydrogen peroxide, a metabolite, an electrolyte, an ion, a drug, a pharmacologic, a biological, or a medicament.

20. The method of claim 17, wherein said infusion system comprises a fluid delivery apparatus configured to provide infusion via a microneedle, microneedle array, macroneedle, hypodermic needle, cannula, catheter, or oral delivery route.
